(19) United States
(12) Patent Application Publication
(10) Pub. No.: US 2007/0185428 A1
(43) Pub. Date: Aug. 9, 2007

(54) Compression bandage with H-anchor tightening means

(76) Inventor: Robert H. Harder, Bena, VA (US)

Correspondence Address: NIXON & VANDERHYE, PC, 901 NORTH GLEBE ROAD, 11TH FLOOR, ARLINGTON, VA, 22203, US

(21) Appl. No.: 11/350,064

(22) Filed: Feb. 9, 2006

(51) Int. Cl. A61L 15/00

(52) U.S. Cl. 602/75

(57) ABSTRACT

Disclosed is a strip of bandaging material having Velcro® strips at either end on opposite sides of the material. An absorbent pad is fixed near one end and on the side opposite the Velcro® strip. On the side opposite the absorbent pad, an S hook is provided with one end fastened to the material and the other end free. The absorbent pad is located over the wound and the longer end of material is wound around the extremity and fixed in place with the Velcro® strip at the near end of the material. The remaining material is passed through the hook and pulled back in order to apply pressure to the wound and reduce blood loss. The remainder of the material is wrapped around the extremity and secured in place with a Velcro® strip and/or a barrette clip on the far end.

### BACKGROUND OF THE INVENTION

1. Field of the Invention

The current compression bandages that are employed to give first aid to trauma wounds are sometimes difficult to adequately tighten securely over the wound. For wounds that are aggressively bleeding, an increase in compression over the wound site is necessary.

2. Discussion of Prior Art

Some past tightening devices that are attached to trauma bandages do not allow a sufficiently large force to be placed on the tightening device to stop arterial bleeding. In addition, some past bandages needed hand tied knots to fasten the bandage to the wound, resulting in loosening of the bandage on the trauma.

A solution to the need for the problem of hand tied knots was the subject matter of U.S. Pat. No. 6,593,508 issued to the current inventor, Robert H. Harder on Jul. 15, 2003 with a divisional of that patent issued Jul. 13, 2004 as U.S. Pat. No. 6,762,338 (hereinafter “the Harder patents”). The Harder patents disclose the structure and method of use of a compression bandage having an S-hook as a tightening means. The passing of the elastic bandage through the open end of the S-hook provides compression on the wound located under the S-hook. While this works extremely well, it is desirable to be able to increase the amount of pressure applied to wounds, particularly arterial bleeding wounds.

### SUMMARY OF THE INVENTION

The compression bandage with an H-anchor tightening means is based on the use of a elastic bandage roll that has a absorbent pad attached near one end, each end of the elastic roll having male Velcro® strips (the trademark for one type of hook and loop fastener system having the male or micro-hook characteristics) to allow closure, in the initial and final application, and an H-shaped anchor attached through both the elastic roll and the absorbent pad attached thereto. The application of the absorbent pad to a trauma seals that wound, and the initial Velcro® strip allows placement of the pad on the wound and retention even if done one-handed.

The elastic roll, after wrapping once around the extremity, is engaged around one leg of the H-anchor sewn into the elastic bandage and absorbent pad. The elastic is then pulled tight and passed around the extremity and is engaged around the other leg of the H-anchor. The elastic bandage is then doubled back on itself again and pulled tight against the H-anchor to give compression to the pad and the wound. Thus each vertical leg of the H is engaged with the elastic bandage pulling on it from both sides of the wound (instead of only one side as in the case of the patented S-hook embodiment. The elastic strip is then stretched and wound around the wound, with the Velcro® strip at the end used to finish fasten.

This invention allows an individual to place a bandage on a wound and, with one hand if necessary, put a higher degree of compression on the wound, easily fastening and securing the bandage.

### BRIEF DESCRIPTION OF THE DRAWINGS

A better understanding of the invention will be had by reference to the following drawings in which:

FIG. 1 is a perspective view of the compression bandage of the present invention;

FIG. 2 is a perspective view showing the initial step of fixing the absorbent pad on the bandage into position over the wound;

FIG. 3 is a perspective view showing the second step of passing the remainder of the bandage around one leg of the H-anchor and applying tension to the bandage;

FIG. 4 is a perspective view showing the third step of passing the remainder of the bandage around the other leg of the H-anchor and then winding the remainder of the bandage around the wound thereby applying additional pressure to the wound; and

FIG. 5 is a perspective view showing the final step of completing the wrapping of the bandage and removably fixing the free end of the bandage in place.

### DETAILED DISCUSSION OF EMBODIMENTS

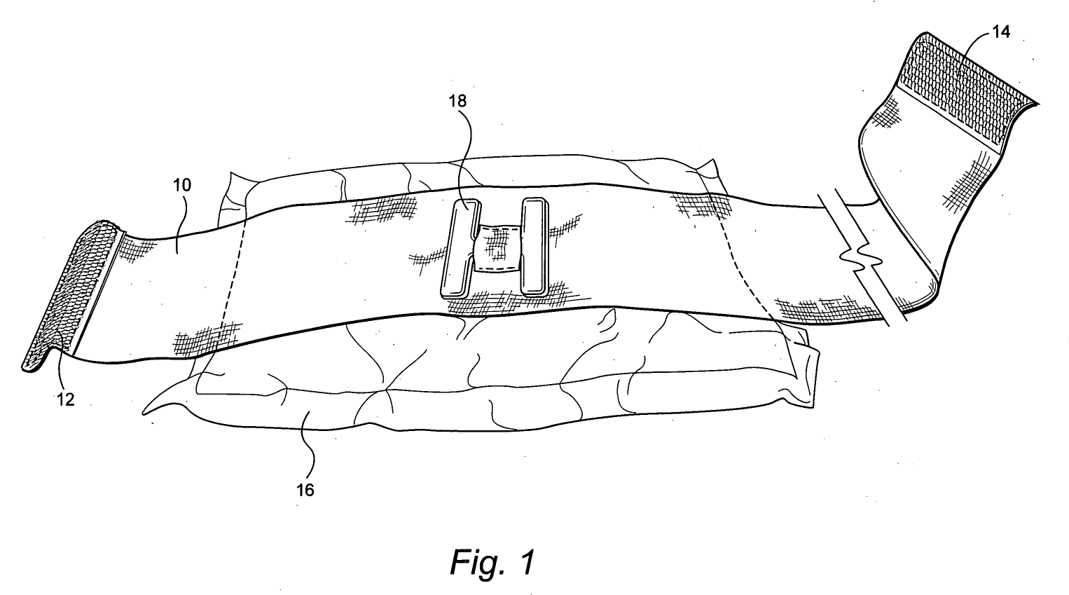

The present invention, an elastic bandage, comprises a strip of material 10 in the form of a roll, preferably at least 3″ to 6″ in width, and 48″ to 60″ long. At each end of the material male Velcro® strips 12, 14 are fastened. Near one end of the elastic roll (the ends of the material are defined as the near end and the far end), a high absorbent pad 16, in a preferred embodiment at least 5″×8,″ is attached. In one embodiment, shown in FIG. 1, at the center of the pad, but on the opposite side of the material from the absorbent pad 16, an anchoring point, in a preferred embodiment, an H-anchor 18 is attached. The H-anchor has two vertical legs located transversely across the bandage and the legs are connected by a cross-piece (forming the letter “H”) which may be fastened to the bandage in any manner, i.e., by gluing, by sewing a transverse tab (as shown), etc.

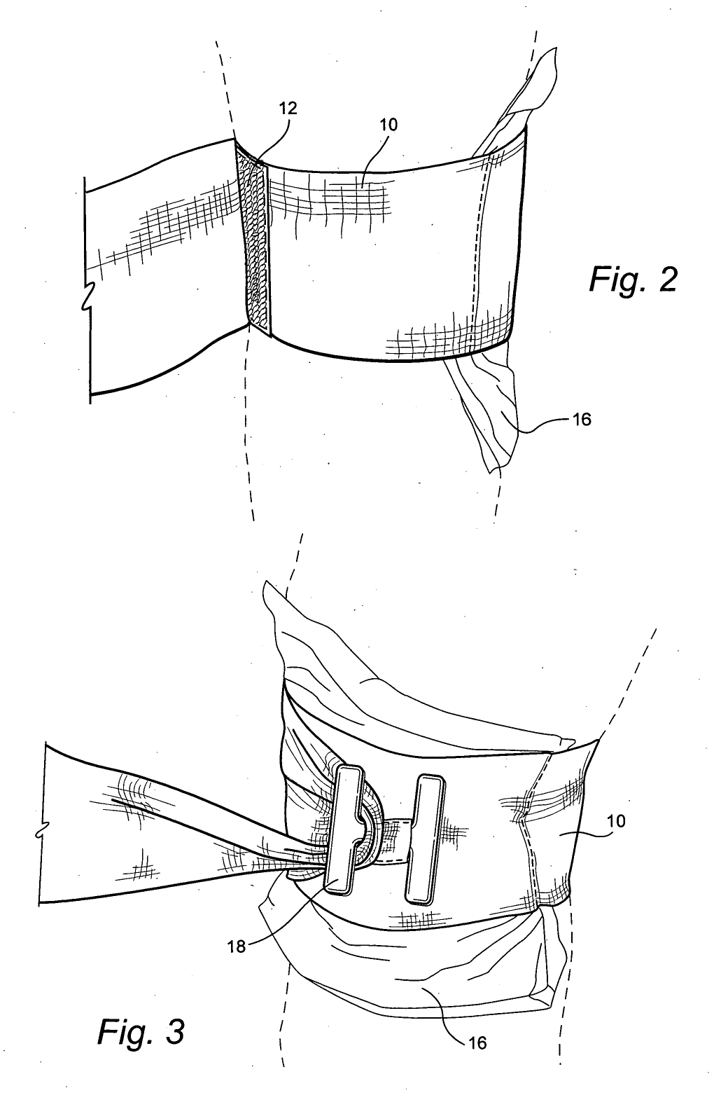

After the pad 16 is located over the wound, the elastic roll is wrapped once around the extremity. The use of the Velcro® strips allows ease of initially holding the pad over the wound site and permits single-handed operation (important for severe wounds in which assistance is not readily available). The Velcro® strip 12 located at the near end of the material may be located on the same side of the material as the H-anchor 18 and initially secures the bandage as the material is initially wrapped around the extremity, with the hooks in the male Velcro® strip engaging the bandage material 10 to temporarily hold the bandage in place as shown in FIG. 2.

The elastic bandage is then passed around one leg of the H-anchor 18 as shown in FIG. 3 and back on itself. It is pulled tight to provide increased tension in the material, thereby tightening the bandage over the wound and at the same time acting as a tourniquet to reduce blood loss. This provides the tension in the elastic bandage and resultant compression on the wound site comparable to the compression achieved by the S-hook anchoring point in the Harder Patents.

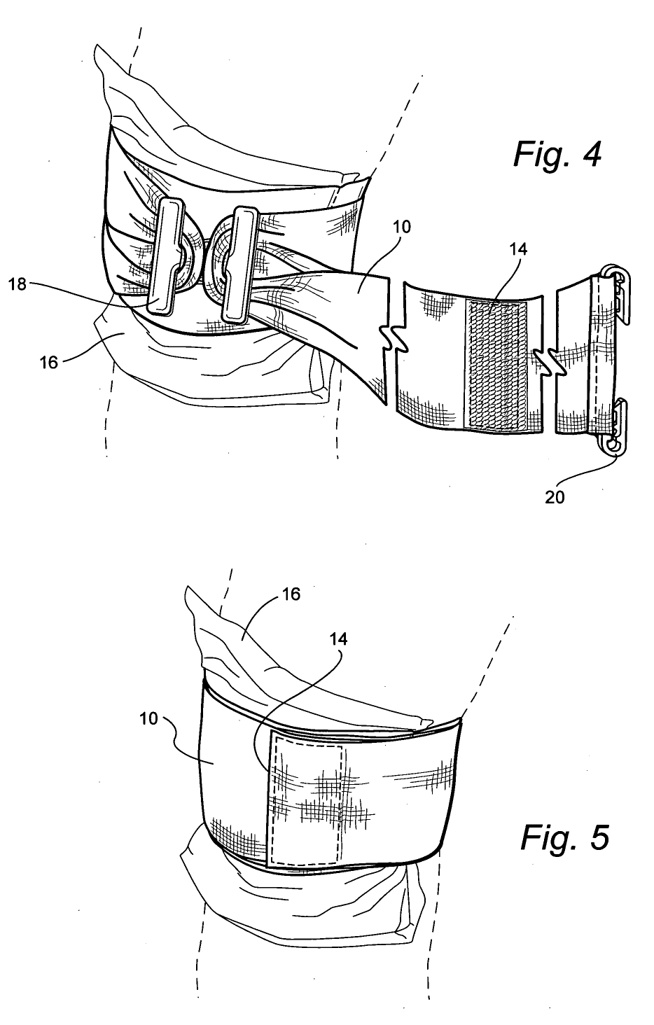

However, as shown in FIG. 4, after passing around the extremity, the elastic bandage is passed around the other leg of the H-anchor and back on itself again. It is pulled tight to retain the increased tension in the material, thereby providing a second force on the H-anchor, further tightening the bandage over the wound. This provides an increase in the tension in the elastic bandage and resultant compression on the wound site over that compression achieved by the S-hook anchoring point in the Harder Patents.

The remaining material is wound over the wound as shown in FIG. 5 and secured in place by engaging the Velcro® strip on the far end of the material with the material itself. Note that in order for the near end Velcro® stripe 12 to engage the material 10 as shown in FIG. 2 it is preferably located on the same side of the material 10 as the H-anchor 18. The far end Velcro® strip 14, in order to engage the material, may be located on either side of the material 10 as twisting of the bandage is likely to occur as it passes around one or both of the H-anchor legs.

Alternately, or in conjunction with the far end Velcro® strip 14, a barrette clip 20 can be sewn into the far end of the material as shown in FIG. 4. Regardless of any twisting of the material, the barrette clip 20 can be rotated so that the teeth capture the upper and lower edges of the underlying layer of material 10, thereby positively securing the end in place.

In one embodiment of the present invention, the H-anchor is made of a high strength, low weight plastic which may include fiber reinforcement, although other materials may be advantageously employed. In one embodiment, the bandage may be sterilized and vacuum packed in a protective plastic casing so as to minimize the necessary storage space.

What is claimed is:

1. An easily applied and secured compression bandage, said bandage comprising:
a bandage comprising a strip of material having first and second sides, said material having relatively near and far ends;
a pad, said pad attached to the first side of said material closer to said near one of said ends;
an anchoring point, attached to the second side of said material, for twice reversing the direction of wrap of said other end of said material, said anchoring point including two legs located in a direction transverse to the bandage; and
a first strip of micro-hooks located on said second side at said near end of said material, said micro-hooks comprising a portion of a hook and loop fastener system, said material comprises a material having loops releasably engageable with said micro-hooks, wherein, when applied, the pad is positioned over a wound on a body or body part, and the far end of said material is passed around the body or body part in one direction of wrap, the material passing over said first strip is fastened thereto temporarily maintaining the position of the pad over the wound, is passed around one leg of the anchoring point and its direction of wrap reversed, is wound around the extremity and passed around the second leg of the anchoring point and is pulled sufficiently tight to deter bleeding and is wound around and fixed to the bandage.

2. The compression bandage according to claim 1, wherein said material is comprised of an elastic material.

3. The compression bandage according to claim 1, wherein said material has a width which is within the range 4 inches to six inches.

4. The compression bandage according to claim 1, wherein said material has a length which is within the range 48 inches to 60 inches.

5. The compression bandage according to claim 1, wherein said pad comprises an absorbent pad.

6. The compression bandage according to claim 5, wherein said absorbent pad comprises a pad having dimensions of at least 5 inches by 8 inches.

7. The compression bandage according to claim 1, wherein said anchoring point comprises an H-shaped anchoring point including a cross-piece joining said two legs, said H-shaped anchoring point is attached on said other side of said material adjacent said pad.

8. The compression bandage according to claim 7, wherein said H-shaped anchoring point is plastic.

9. The compression bandage according to claim 1, wherein said anchoring point is located at the center of the pad but on the opposite side of the material from the pad.

10. The compression bandage according to claim 1, further including a second strip of micro-hooks located near said far end of said material for securing said far end to said material wound around said extremity.

11. The compression bandage according to claim 1, further including a barrette sew into the far end of said material for securing said far end to said material wound around said extremity.

12. An easily applied and secured compression bandage, said bandage comprising:
a bandage comprising a strip of material having first and second sides, said material having relatively near and far ends;
a pad, said pad attached to the first side of said material closer to said near one of said ends;
an H-shaped anchoring point, attached to the second side of said material, for twice reversing the direction of wrap of said other end of said material, said H-shaped anchoring point including two legs located in a direction transverse to the bandage; and
a first strip of micro-hooks located on said second side at said near end of said material, said micro-hooks comprising a portion of a hook and loop fastener system, said material comprises a material having loops releasably engageable with said micro-hooks, wherein, when applied, the pad is positioned over a wound on a body or body part, and the far end of said material is passed around the body or body part in one direction of wrap, the material passing over said first strip is fastened thereto temporarily maintaining the position of the pad over the wound, is passed around one leg of the H-shaped anchoring point and its direction of wrap reversed, is wound around the extremity and passed around the second leg of the H-shaped anchoring point and is pulled sufficiently tight to deter bleeding and is wound around and fixed to the bandage.

13. The compression bandage according to claim 12, wherein said material is comprised of an elastic material.

14. The compression bandage according to claim 12, wherein said material has a width which is within the range 4 inches to six inches.

15. The compression bandage according to claim 12, wherein said material has a length which is within the range 48 inches to 60 inches.

16. The compression bandage according to claim 12, wherein said pad comprises an absorbent pad.

17. The compression bandage according to claim 16, wherein said absorbent pad comprises a pad having dimensions of at least 5 inches by 8 inches.

18. The compression bandage according to claim 12, wherein said H-shaped anchoring point includes a cross-piece joining said two legs, said H-shaped anchoring point is attached on said other side of said material adjacent said pad and said top being free.

19. The compression bandage according to claim 18, wherein said H-shaped anchoring point is plastic.

20. The compression bandage according to claim 12, wherein said anchoring point is located at the center of the pad but on the opposite side of the material from the pad.

21. An easily applied and secured compression bandage, said bandage comprising:
a bandage comprising a strip of material having first and second sides, said material having relatively near and far ends;
a pad, said pad attached to the first side of said material closer to said near one of said ends; and
an anchoring point, attached to the second side of said material, for twice reversing the direction of wrap of said other end of said material, said anchoring point including two legs located in a direction transverse to the bandage.

22. An easily applied and secured compression bandage, said bandage comprising:
a bandage comprising a strip of material having first and second sides, said material having relatively near and far ends;
a pad, said pad attached to the first side of said material closer to said near one of said ends;
an H-shaped anchoring point, attached to the second side of said material, for twice reversing the direction of wrap of said other end of said material, said H-shaped anchoring point including two legs located in a direction transverse to the bandage; and
a first strip of micro-hooks located on said second side at said near end of said material, said micro-hooks comprising a portion of a hook and loop fastener system, said material comprises a material having loops releasably engageable with said micro-hooks.

23. The compression bandage according to claim 12, further including a second strip of micro-hooks located near said far end of said material for securing said far end to said material wound around said extremity.

24. The compression bandage according to claim 12, further including a barrette sew into the far end of said material for securing said far end to said material wound around said extremity.
